(19) United States
(12) Patent Application Publication
(10) Pub. No.: US 2006/0051368 A1
(43) Pub. Date: Mar. 9, 2006

(54) Herpes simplex virus mutations

(75) Inventors: Patricia G. Spear, Chicago, IL (US); Sharmila Manoj, Arlington Heights, IL (US); Cheryl R. Jogger, Boston, MA (US); Dawn Myscofski, Washington, DC (US); Miri Yoon, Chicago, IL (US)

Correspondence Address: David A. Casimir;MEDLEN & CARROLL, LLP, Suite 350, 101 Howard Street, San Francisco, CA, 94105, US

(73) Assignee: Northwestern University, Evanston, IL (US)

(21) Appl. No.: 11/187,652

(22) Filed: Jul. 22, 2005

Related U.S. Application Data
(60) Provisional application No. 60/590,729, filed on Jul. 23, 2004.

(51) Int. Cl. A61K 39/12; C07H 21/04; C07K 14/035; C12N 15/86

(52) U.S. Cl. 424/199.1; 435/69.3; 435/456; 435/325; 530/350; 536/23.72

(57) ABSTRACT

The present invention relates to compositions and methods related to mutant herpes simplex virus nucleic acid and proteins that find use in analyzing, diagnosing, and regulating viral infection.

This application claims priority to U.S. provisional application No. 60/590,729 filed Jul. 23, 2004, which is hereby incorporated herein by reference in its entirety.

The present invention was funded in part under U.S. Public Health Service grants CA-21776, AI-31494, AI-36293 and AI53774. The government may have certain rights in the invention.

### FIELD OF THE INVENTION

The present invention relates to compositions and methods related to mutant herpes simplex virus nucleic acid and proteins that find use in analyzing, diagnosing, and regulating viral infection.

### BACKGROUND OF THE INVENTION

The usual manifestations of human disease caused by herpes simplex viruses (HSV) are mucocutaneous lesions, resulting from replication of virus in epithelial cells of skin or mucosa and influx of leukocytes, which may also become infected. These lesions may be mild or severe, but can recur due to the establishment of latent infections in sensory neurons and subsequent reactivation of replicating virus. Rare manifestations of HSV disease include encephalitis, meningitis, and disseminated disease affecting multiple organ systems. If HSV were unable to invade neurons, the most serious consequences of disease would probably not occur and the latent infections leading to life-long HSV persistence would likely not occur.

Entry receptors for HSV include human and animal members of three classes of cell surface molecules (Spear, P. G., Eisenberg, R. J. & Cohen, G. H. (2000) Virology 275, 1-8.). The herpesvirus entry mediator (HVEM or HveA) (Montgomery, R. I., Warner, M. S., Lum, B. J. & Spear, P. G. (1996) Cell 87, 427-436.), a member of the tumor necrosis factor receptor family, is widely expressed and has a role in the regulation of immune responses (Granger, S. W. & Rickert, S. (2003) Cytokine Growth Factor Rev 14, 289-296.). Nectin-1 (Prr1 or HveC) (Geraghty, R. J., Krummenacher, C., Cohen, G. H., Eisenberg, R. J. & Spear, P. G. (1998) Science 280, 1618-1620, Cocchi, F., Menotti, L., Mirandola, P., Lopez, M. & Campadelli-Fiume, G. (1998) J. Virol. 72, 9992-10002. and nectin-2 (Prr2 or HveB) (Warner, M. S., Geraghty, R. J., Martinez, W. M., Montgomery, R. I., Whitbeck, J. C., Xu, R., Eisenberg, R. J., Cohen, G. H. & Spear, P. G. (1998) Virology 246, 179-189.), members of the immunoglobulin superfamily related to the poliovirus receptor, are cell adhesion molecules that can participate in cadherin-based and other cell junctions, including junctions in the nervous system, and are widely expressed in many organs and tissues (Takai, Y., Irie, K., Shimizu, K., Sakisaka, T. & Ikeda, W. (2003) Cancer Sci 94, 655-67.). Certain isoforms of 3-O-sulfotransferases, such as 30ST-3A, 3-OST-3B, and 3-OST-5 generate sites in heparan sulfate (3-O-S HS) that can serve as HSV entry receptors (Shukla, D., Liu, J., Blaiklock, P., Shworak, N. W., Bai, X., Esko, J. D., Cohen, G. H., Eisenberg, R. J., Rosenberg, R. D. & Spear, P. G. (1999) Cell 99, 13-22, Xia, G., Chen, J., Tiwari, V., Ju, W., L1, J.-P., Malmstrom, A., Shukla, D. & Liu, J. (2002) J. Biol. Chem. 277, 37912-37919.), whereas others generate sites that can bind to antithrombin to enhance anti-coagulation (Xia, G., Chen, J., Tiwari, V., Ju, W., L1, J.-P., Malmstrom, A., Shukla, D. & Liu, J. (2002) J. Biol. Chem. 277, 37912-37919, Liu, J., Shworak, N. W., Sinay, P., Schwartz, J. J., Zhang, L., Fritze, L. M. S. & Rosenberg, R. (1999) J. Biol. Chem. 274, 5185-5192.). This variety of entry receptors may allow HSV to use different receptors for infection of various target cell types.

There are two serotypes of HSV (HSV-1 and HSV-2) that differ in natural history of disease and in entry receptor preferences. HSV-1 is the usual cause of oral and corneal lesions and adult encephalitis, whereas HSV-2 is more likely to be the cause of genital herpes, meningitis and neonatal herpes acquired during delivery. Nectin-1 and HVEM are entry receptors for both HSV-1 and HSV-2 strains, whereas nectin-2 is a better receptor for HSV-2 than for HSV-1 (Warner, M. S., Geraghty, R. J., Martinez, W. M., Montgomery, R. I., Whitbeck, J. C., Xu, R., Eisenberg, R. J., Cohen, G. H. & Spear, P. G. (1998) Virology 246, 179-189.), except for some HSV-1 strains isolated from cases of encephalitis (Krummenacher, C., Baribaud, F., Ponce de Leon, M., Baribaud, I., Whitbeck, J. C., Xu, R., Cohen, G. H. & Eisenberg, R. J. (2004) Virology 322, 286-299.). On the other hand, 3-O-S HS appears to be a better receptor for HSV-1 than for HSV-2 (Shukla, D., Liu, J., Blaiklock, P., Shworak, N. W., Bai, X., Esko, J. D., Cohen, G. H., Eisenberg, R. J., Rosenberg, R. D. & Spear, P. G. (1999) Cell 99, 13-22.).

Four HSV glycoproteins (gB, gD, gH and gL) are essential for viral entry (Spear, P. G. (1993) Sem. Virol. 4, 167-180.), and are both necessary and sufficient for HSV-induced cell fusion (Turner, A., Bruun, B., Minson, T. & Browne, H. (1998) J. Virol. 72, 873-875.). The viral ligand for all the entry receptors described above is gD (Shukla, D., Liu, J., Blaiklock, P., Shworak, N. W., Bai, X., Esko, J. D., Cohen, G. H., Eisenberg, R. J., Rosenberg, R. D. & Spear, P. G. (1999) Cell 99, 13-22, Whitbeck, J. C., Peng, C., Lou, H., Xu, R., Willis, S. H., Ponce de Leon, M., Peng, T., Nicola, A. V., Montgomery, R. I., Warner, M. S., Soulika, A. M., Spruce, L. A., Moore, W. T., Lambris, J. D., Spear, P. G., Cohen, G. H. & Eisenberg, R. J. (1997) J. Virol. 71, 6083-6093, Krummenacher, C., Nicola, A. V., Whitbeck, J. C., Lou, H., Hou, W., Lambris, J. D., Geraghty, R. J., Spear, P. G., Cohen, G. H. & Eisenberg, R. J. (1998) J. Virol. 72, 7064-7074, Cocchi, F., Lopez, M., Menotti, L., Aoubala, M., Dubreuil, P. & Campadelli-Fiume, G. (1998) Proc. Natl. Acad. Sci. U.S.A. 95, 15700-15705.). Interaction of gD with one of its receptors triggers the fusogenic activity of one of the other essential HSV glycoproteins (Spear, P. G. & Longnecker, R. (2003) J. Virol. 77, 10179-10185, Cocchi, F., Fusco, D., Menotti, L., Gianni, T., Eisenberg, R. J., Cohen, G. H. & Campadelli-Fiume, G. (2004) Proc. Natl. Acad. Sci. U.S.A. 101, 7445-7450.).

Information about structural requirements for the interactions of gD with some of its receptors has come from X-ray crystallography and mutational analysis of gD. X-ray structures were determined for a large portion of the HSV-1 gD ectodomain, either crystallized alone or in complex with the N-terminal two cysteine-rich domains of HVEM (Carfi, A., Willis, S. H., Whitbeck, J. C., Krunimmenacher, C., Cohen, G. H., Eisenberg, R. J. & Wiley, D. C. (2001) Mol. Cell 8, 169-179.). Direct physical contact of HVEM with gD has been localized to an N-terminal hairpin in gD and involved amino acids 7-15 and 24-32. This N-terminal region was extended and partly disordered when the gD ectodomain was crystallized alone. Certain amino acid substitutions or insertions within the first 43 amino acids of HSV-1 or HSV-2 gD abrogate physical and functional interactions with HVEM (Montgomery, R. I., Warner, M. S., Lum, B. J. & Spear, P. G. (1996) Cell 87, 427-436, Whitbeck, J. C., Peng, C., Lou, H., Xu, R., Willis, S. H., Ponce de Leon, M., Peng, T., Nicola, A. V., Montgomery, R. I., Warner, M. S., Soulika, A. M., Spruce, L. A., Moore, W. T., Lambris, J. D., Spear, P. G., Cohen, G. H. & Eisenberg, R. J. (1997) J. Virol. 71, 6083-6093, Connolly, S. A., Landsburg, D. J., Carfi, A., Wiley, D. C., Cohen, G. H. & Eisenberg, R. J. (2003) J. Virol. 77, 8127-8140, Jogger, C. R., Montgomery, R. I. & Spear, P. G. (2004) Virology 318, 318-326, Yoon, M., Zago, A., Shukla, D. & Spear, P. G. (2003) J. Virol. 77, 9221-9231.) consistent with the X-ray structure of the gD-HVEM complex. Some of these mutations near the N-terminus also prevent functional interactions of HSV-1 gD with 3-O-S HS (Yoon, M., Zago, A., Shukla, D. & Spear, P. G. (2003) J. Virol. 77, 9221-9231.), and may confer ability to use nectin-2 as an entry/fusion receptor (Warner, M. S., Geraghty, R. J., Martinez, W. M., Montgomery, R. I., Whitbeck, J. C., Xu, R., Eisenberg, R. J., Cohen, G. H. & Spear, P. G. (1998) Virology 246, 179-189, Connolly, S. A., Landsburg, D. J., Carfi, A., Wiley, D. C., Cohen, G. H. & Eisenberg, R. J. (2003) J. Virol. 77, 8127-8140, Yoon, M., Zago, A., Shukla, D. & Spear, P. G. (2003) J. Virol. 77, 9221-9231, Lopez, M., Cocchi, F., Menotti, L., Avitabile, E., Dubreuil, P. & Campadelli-Fiume, G. (2000) J. Virol. 74, 1267-1274.). Deletion of subsets or all amino acids from positions 7-32 in either HSV-1 or HSV-2 gD abrogated cell fusion activity with all known receptors except nectin-1, leading to the conclusion that the major contact site for nectin-1 must be downstream of the HVEM-binding domain (Yoon, M., Zago, A., Shukla, D. & Spear, P. G. (2003) J. Virol. 77, 9221-9231.). This is in all likelihood true also for nectin-2, because nectin-1 and nectin-2 are closely related and homologous regions of both are critical for functional interactions with gD (Martinez, W. M. & Spear, P. G. (2001) J. Virol. 75, 11185-11195, Martinez, W. M. & Spear, P. G. (2002) J. Virol. 76, 7255-7262.). To date, no mutations in gD have been described that abrogate functional interactions with nectin-1 without also affecting activities with other entry receptors such as HVEM (Jogger, C. R., Montgomery, R. I. & Spear, P. G. (2004) Virology 318, 318-326.).

### SUMMARY OF THE INVENTION

The present invention relates to compositions and methods related to mutant herpes simplex virus nucleic acid and proteins that find use in analyzing, diagnosing, and regulating viral infection.

Experiments conducted during the development of the present invention have identified mutations in the HSV gD ectodomain that abrogate functional interactions with nectin-1 without also affecting activities with other entry receptors such as HVEM, that alter activity with nectin-2, and that reduce or abolish entry/fusion activity when a nectin is required for infection of selected cell types. It has been found that double or triple amino acid substitutions at positions 215, 222, 223, and single mutations at position 38 reduce or abolish entry/fusion activity with nectin-1 and nectin-2 without preventing activity with HVEM or 3-O-S HS. Viruses containing these mutant forms of gD are unable to infect human cell lines of neuronal or epithelial origin whereas viruses containing gD mutants active with nectin-1, but not with any of the other receptors, are able to infect the cells. Although an understanding of the mechanism is not necessary to practice the present invention, and the present invention is not limited to any particular mechanism of action, nectin-1 and nectin-2, may be the principal receptors for HSV entry into neurons, and mutant viruses unable to use nectins for intracellular entry could spare neurons from infection,

Thus, the present invention provides compositions and methods related to mutant herpes simplex virus nucleic acid and proteins that find use in analyzing, diagnosing, and regulating viral infection. Analysis and diagnostic uses include research into viral infection function, as well as drug screening methods (e.g., for drugs that prevent or otherwise regulate viral entry or viral interaction with cellular proteins). Regulation uses include research and therapeutic uses, for example, involving compounds (e.g., antibodies, small molecules, proteins, aptamers, nucleic acids, gene therapy, etc.) that regulate viral entry, mutated entry-deficient viruses or portions of viruses for use as vaccines, transgene vectors, and the like.

### DESCRIPTION OF THE DRAWINGS

FIG. 1 shows binding of HSV-1 and HSV-2 wild-type and mutant gD:Fcs to CHO-K1 cells expressing human forms of HVEM and nectin-1.

FIG. 2 shows binding of HSV-1 wild-type and mutant gD:Fcs to CHO cells expressing 3-OST-3A (top panel), and cell fusion activity of wild-type and mutant. HSV-1 gD with target CHO cells expressing 3-OST-3A (bottom panel).

FIG. 3 shows cell fusion activities of HSV-1 and HSV-2 gD mutants with target BHK-95-19 cells expressing HVEM, nectin-1 or nectin-2.

FIG. 4 shows viral entry activities of the HSV-1 and HSV-2 gD mutants in CHO-HVEM and CHO-nectin-1 cells.

FIG. 5 shows viral entry activities of the HSV-1 and HSV-2 gD mutants in human neuroblastoma cell lines, IMR-5 and SH-SY5Y.

FIG. 6 shows viral entry activities of the HSV-2 gD mutants in two human epithelial cell lines, A431 and C33A, and in the Jurkat T cell line.

FIG. 7 shows locations of the critical amino acid substitutions (D215, R222, F223) in gD crystallized alone (top) and gD crystallized with HVEM (bottom—HVEM is not shown).

FIG. 8 shows the effects of mutations in herpes simplex virus type 1 (HSV-1) gD on viral entry into cells expressing three different viral entry receptors, HVEM, nectin-1 or nectin-2.

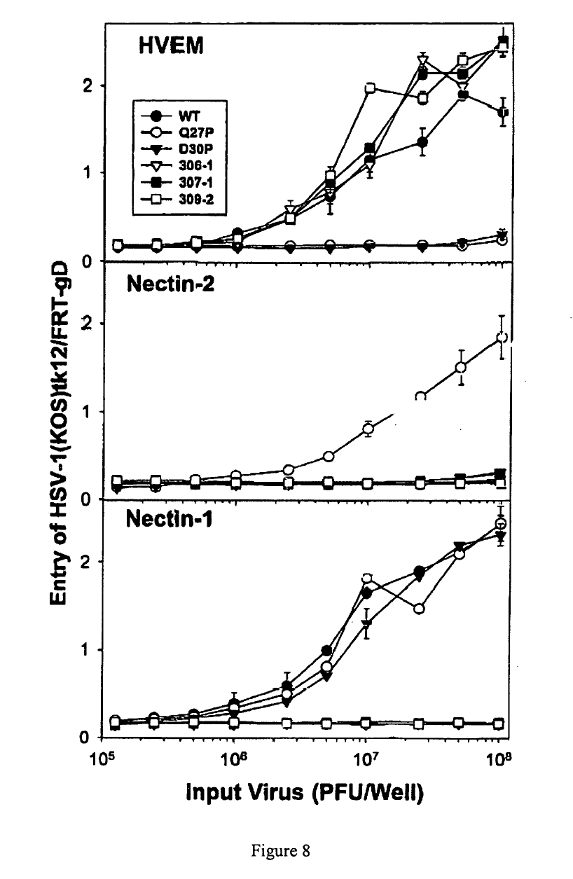

### DEFINITIONS

As used herein, the term “nuclear targeting signal” refers to a nucleic acid sequence, that, when operably linked to a nucleic acid sequence of interest, directs import of the nucleic acid into the nucleus of cell. In certain embodiments, the nuclear targeting signal further directs the expression of the nucleic acid of interest in the nucleus. In particularly preferred embodiments, the nuclear targeting signal is “cell specific.”

As used herein, “cell-specific” means the targeting of DNA to the nuclei of a specific cell type or types of interest only, and not to the nuclei of other cell types. The “specific cell type” refers to a “type” of cell (for example, pulmonary epithelial cells). In further embodiments, “substantially cell-specific” means the preferential targeting of DNA to the nuclei of a specific cell type or types more so than to the nuclei of other cells types.

As used herein, “nuclear DNA binding proteins” refer to DNA binding proteins that reside in the nucleus. These nuclear DNA binding proteins are characterized in that they bind to short DNA sequences with sequence specificity, and they are transported to the nucleus of a cell because they contain a nuclear localization signal (NLS) or because they complex with one or more other proteins that contain an NLS. Nuclear DNA binding proteins have different functions in the regulation of DNA transcription and/or replication. Nuclear DNA binding proteins include, for example, eukaryotic transcription factors, DNA replication factors, and telomere or centromere binding proteins. For a general discussion of nuclear, DNA binding proteins, see e.g., Nigg (Nature 386:779 (1997)). Preferably, the nuclear DNA binding protein is a transcription factor.

As used herein, “transcription factors” refer to proteins that promote RNA polymerase recognition and/or initiation and/or activation and/or repression of promoters (DNA sequences). The binding of RNA polymerase to a promoter is important to initiate transcription, which is the process by which the information contained in the DNA is copied into a single-stranded RNA molecule by RNA polymerase. The genetic information present in a mRNA molecule is then translated into a protein.

The term “gene” refers to a nucleic acid (e.g., DNA or RNA) sequence that comprises coding sequences necessary for the production of a polypeptide or precursor (e.g., a protein of interest). The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb on either end such that the gene corresponds to the length of the full-length mRNA. The sequences that are located 5′ of the coding region and which are present on the mRNA are referred to as 5′ untranslated sequences. The sequences that are located 3′ or downstream of the coding region and that are present on the mRNA are referred to as 3′ untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “heterologous gene” refers to a gene that is not in its natural environment. For example, a heterologous gene includes a gene from one species introduced into another species. A heterologous gene also includes a gene native to an organism that has been altered in some way (e.g., mutated, added in multiple copies, linked to non-native regulatory sequences, etc). Heterologous genes are distinguished from endogenous genes in that the heterologous gene sequences are typically joined to DNA sequences that are not found naturally associated with the gene sequences in the chromosome or are associated with portions of the chromosome not found in nature (e.g., genes expressed in loci where the gene is not normally expressed). As used herein, “heterologous nucleic acid sequence” refers to a nucleic acid sequence that is not in its natural environment.

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified”, “mutant”, and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

A gene may produce multiple RNA species that are generated by differential splicing of the primary RNA transcript. cDNAs that are splice variants of the same gene will contain regions of sequence identity or complete homology (representing the presence of the same exon or portion of the same exon on both cDNAs) and regions of complete non-identity (for example, representing the presence of exon “A” on cDNA 1 wherein cDNA 2 contains exon “B” instead). Because the two cDNAs contain regions of sequence identity they will both hybridize to a probe derived from the entire gene or portions of the gene containing sequences found on both cDNAs; the two splice variants are therefore substantially homologous to such a probe and to each other.

The term “fragment” as used herein refers to a polypeptide that has an amino-terminal and/or carboxy-terminal deletion as compared to the native protein, but where the remaining amino acid sequence is identical to the corresponding positions in the amino acid sequence deduced from a full-length cDNA sequence. Fragments typically are at least 4 amino acids long, preferably at least 20 amino acids long, usually at least 50 amino acids long or longer, and span the portion of the polypeptide required for intermolecular binding of the compositions(claimed in the present invention) with its various ligands and/or substrates.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

The term “transfection” as used herein refers to the introduction of foreign DNA into eukaryotic cells. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The term “stable transfectant” refers to a cell that has stably integrated foreign DNA into the genomic DNA.

The term “transient transfection” or “transiently transfected” refers to the introduction of foreign DNA into a cell where the foreign DNA fails to integrate into the genome of the transfected cell. During the time the foreign DNA persists in the nucleus it is subject to the regulatory controls that govern the expression of endogenous genes in the chromosomes. The term “transient transfectant” refers to cells that have taken up foreign DNA but have failed to integrate this DNA.

The term “calcium phosphate co-precipitation” refers to a technique for the introduction of nucleic acids into a cell. The uptake of nucleic acids by cells is enhanced when the nucleic acid is presented as a calcium phosphate-nucleic acid co-precipitate. The original technique of Graham and van der Eb (Graham and van der Eb, (1973) Virol., 52:456), has been modified by several groups to optimize conditions for particular types of cells. The art is well aware of these numerous modifications.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

As used herein, the term “response,” when used in reference to an assay, refers to the generation of a detectable signal (e.g., accumulation of reporter protein, increase in ion concentration, accumulation of a detectable chemical product).

As used herein, the term “membrane protein” refers to a protein that spans the lipid by-layer membrane or a cell or organelle.

As used herein, the term “ion channel protein” refers to proteins that control the ingress or egress of ions across cell membranes. Examples of ion channel proteins include, but are not limited to, the Na⁺-K⁺ ATPase pump, the Ca²⁺ pump, and the K⁺ leak channel.

As used herein, the term “purified” refers to molecules, either nucleic or amino acid sequences that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated.

As used herein, the term “siRNAs” refers to small interfering RNAs. In some embodiments, siRNAs comprise a duplex, or double-stranded region, of about 18-25 nucleotides long; often siRNAs contain from about two to four unpaired nucleotides at the 3′ end of each strand. At least one strand of the duplex or double-stranded region of a siRNA is substantially homologous to, or substantially complementary to, a target RNA molecule. The strand complementary to a target RNA molecule is the “antisense strand;” the strand homologous to the target RNA molecule is the “sense strand,” and is also complementary to the siRNA antisense strand. siRNAs may also contain additional sequences; non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing in plants.

The term “RNA interference” or “RNAi” refers to the silencing or decreasing of gene expression by siRNAs. It is the process of sequence-specific, post-transcriptional gene silencing in animals and plants, initiated by siRNA that is homologous in its duplex region to the sequence of the silenced gene. The gene may be endogenous or exogenous to the organism, present integrated into a chromosome or present in a transfection vector that is not integrated into the genome. The expression of the gene is either completely or partially inhibited. RNAi may also be considered to inhibit the function of a target RNA; the function of the target RNA may be complete or partial.

### DETAILED DESCRIPTION OF THE INVENTION

The present invention relates to compositions and methods related to mutant herpes simplex virus nucleic acid and proteins that find use in analyzing, diagnosing, and regulating viral infection.

Glycoprotein D (gD) determines which cells can be infected by herpes simplex virus (HSV) by binding to one of the several cell surface receptors that can mediate HSV entry or cell fusion. These receptors include the herpesvirus entry mediator (HVEM), nectin-1, nectin-2 and sites in heparan sulfate generated by specific 3-O-sulfotransferases. The present invention provides mutations (e.g., double or triple amino acid substitutions at positions 215, 222 and 223 functional equivalents, and single amino acid substitutions at position 38) in gD that cause marked reduction in gD binding to nectin-1 and a corresponding inability to function in cell fusion or entry of HSV via nectin-1 or nectin-2. These substitutions either enhanced, or did not significantly inhibit, functional interactions with HVEM and modified heparan sulfate. These and other results demonstrate that different domains of gD, with some overlap, are important for functional interactions with each class of entry receptor. Viral entry assays, using gD mutants described here and previously, revealed that nectins are the principal entry receptors for selected human cell lines of neuronal and epithelial origin whereas HVEM or nectins could be used to mediate entry into a T lymphocyte line. Because T cells and fibroblasts can be infected via HVEM, HSV strains carrying gD mutations that prevent entry via nectins are contemplated to establish transient infections in humans, but not latent infections of neurons, and therefore provide safe live virus vaccines and vaccine vectors.

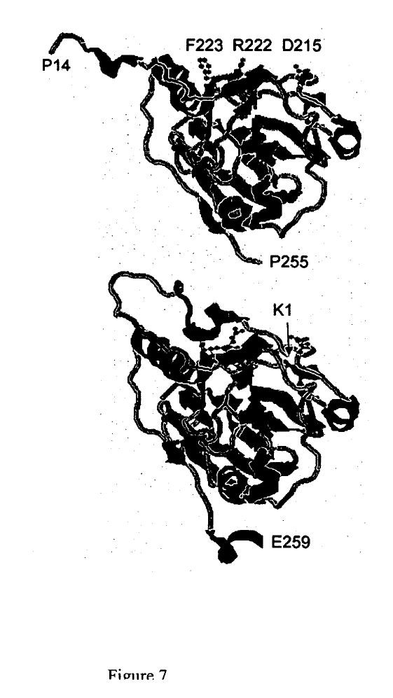

Experiments conducted in the course of development of the present invention, when considered with the X-ray structures of gD (Carfi, A., Willis, S. H., Whitbeck, J. C., Krummenacher, C., Cohen, G. H., Eisenberg, R. J. & Wiley, D. C. (2001) Mol. Cell 8, 169-179.), identify a binding site on gD for the nectins and provide additional evidence that HVEM and the nectins bind to different regions of gD. FIG. 7 presents two models of the gD ectodomain, although an understanding of the mechanism is not necessary to practice the present invention and the present invention is not limited to any particular mechanism of action. Beta-strands are colored light and alpha-helices dark except for the contact sites with HVEM (amino acids 7-15 and 24-32), which are intermediately colored. The entire ectodomain of gD is about 316 amino acids. A truncated form of gD used for crystallization (amino acids 1-285) yielded structures in which only the first 255 (minus 1-13) or 259 amino acids were visible. The structures shown are based on the coordinates deposited in the Protein Data Bank (Berman, H. M., Westbrook, J., Feng, Z., Gilliland, G., Bhat, T. M., Weissig, H., Shindyalov, I. N. & Boume, P. E. (2000) Nucl. Acids Res. 28, 235-242.) for entries 1JMA and 1L2G (Carfi, A., Willis, S. H., Whitbeck, J. C., Krummenacher, C., Cohen, G. H., Eisenberg, R. J. & Wiley, D. C. (2001) Mol. Cell 8, 169-179.). Molecular graphics images were produced using the UCSF CHIMERA package (Huang C. C., Couch G. S., Pettersen, E. F., Ferrin T. E. (1996) CHIMERA, An Extensible Molecular Modeling Application Constructed Using Standard Components (Computer Graphics Lab., Univ. of Calif., San Francisco, Calif.). One model is for gD crystallized alone and the other is for the complex of HVEM with gD. The positions of the amino acid substitutions (R222N/F223I/D215G) that impaired most severely the binding and activity of gD with nectin-1 and nectin-2 are indicated on both models. Note that in gD crystallized alone, these residues are accessible to solvent and lie together on an exposed surface that could theoretically bind to the nectins. These residues, particularly F223, are exposed only because the N-terminus of gD is extended (the first 13 amino acids are disordered and not shown). In the structure of gD complexed with HVEM, the N-terminus assumes a hairpin turn that folds over onto the region of interest, making F223 much less accessible to solvent. These considerations raise the possibility that gD assumes one conformation when bound to HVEM and may have to assume another conformation upon binding to nectin-1 or nectin-2, a possibility previously mentioned by Carfi et al. (Carfi, A., Willis, S. H., Whitbeck, J. C., Krummenacher, C., Cohen, G. H., Eisenberg, R. J. & Wiley, D. C. (2001) Mol. Cell 8, 169-179.), although an understanding of the mechanism is not necessary to practice the present invention and the present invention is not limited to any particular mechanism of action.

Clearly, the principal contact sites on gD for binding to HVEM and nectin-1 are different. The X-ray structure of the HVEM-GD complex revealed that the amino acids of gD directly in contact with HVEM included residues 7-15 and 24-32 in the N-terminal hairpin (Carfi, A., Willis, S. H., Whitbeck, J. C., Krummenacher, C., Cohen, G. H., Eisenberg, R. J. & Wiley, D. C. (2001) Mol. Cell 8, 169-179.). Amino acid substitutions and deletions that abrogate functional interactions with HVEM lie within this region (Montgomery, R. I., Warner, M. S., Lum, B. J. & Spear, P. G. (1996) Cell 87, 427-436, Whitbeck, J. C., Peng, C., Lou, H., Xu, R., Willis, S. H., Ponce de Leon, M., Peng, T., Nicola, A. V., Montgomery, R. I., Warner, M. S., Soulika, A. M., Spruce, L. A., Moore, W. T., Lambris, J. D., Spear, P. G.; Cohen, G. H. & Eisenberg, R. J. (1997) J. Virol. 71, 6083-6093, Connolly, S. A., Landsburg, D. J., Carfi, A., Wiley, D. C., Cohen, G. H. & Eisenberg, R. J. (2003) J. Virol. 77, 8127-8140, Jogger, C. R., Montgomery, R. I. & Spear, P. G. (2004) Virology 318, 318-326, Yoon, M., Zago, A., Shukla, D. & Spear, P. G. (2003) J. Virol. 77, 9221-9231.). On the other hand, amino acid substitutions at positions Y38, D215, R222 and F223 abrogate physical and functional interactions with nectin-1 and nectin-2 but have little or no effect on such interactions with HVEM.

It should be noted that single amino acid substitutions in the N-terminal domain of HSV-1 gD can confer ability to use nectin-2 as an entry/fusion receptor (Warner, M. S., Geraghty, R. J., Martinez, W. M., Montgomery, R. I., Whitbeck, J. C., Xu, R., Eisenberg, R. J., Cohen, G. H. & Spear, P. G. (1998) Virology 246, 179-189, Connolly, S. A., Landsburg, D. J., Carfi, A., Wiley, D. C., Cohen, G. H. & Eisenberg, R. J. (2003) J. Virol. 77, 8127-8140, Yoon, M., Zago, A., Shukla, D. & Spear, P. G. (2003) J. Virol. 77, 9221-9231, Lopez, M., Cocchi, F., Menotti, L., Avitabile, E., Dubreuil, P. & Campadelli-Fiume, G. (2000) J. Virol. 74, 1267-1274.), and that deletion of amino acids 7-32 abrogates the ability of HSV-2 gD to interact functionally with nectin-2 but is without effect on interactions with nectin-1, for either HSV-1 or HSV-2 gD (Yoon, M., Zago, A., Shukla, D. & Spear, P. G. (2003) J. Virol. 77, 9221-9231.). The N-terminal region of HSV-1 or HSV-2 gD influences whether a functional interaction with nectin-2, but not nectin-1, can occur. Perhaps the N-terminus provides a secondary contact site for nectin-2, or influences the conformation of the primary contact site in a manner that is critical for nectin-2 binding but irrelevant for nectin-1 binding, although an understanding of the mechanism is not necessary to practice the present invention and the present invention is not limited to any particular mechanism of action.

A surprising effect of the amino acid substitutions at positions D215, R222 and F223 (double and triple mutations) is the significant enhancement of the ability of HSV-1 gD to bind to 3-O-S HS, and to use sites in this modified heparan sulfate as cell fusion receptors. Mutations in the N-terminus of HSV-1 gD (L25P, Q27P/R or A7-32) abrogate functional interactions with 3-O-S HS (Yoon, M., Zago, A., Shukla, D. & Spear, P. G. (2003) J. Virol. 77, 9221-9231.). These latter mutations are near a positively-charged deep pocket in the gD structure that is occupied by an anion (probably a sulfate ion) and is suggested to be a potential binding site for 3-O-S HS (Carfi, A., Willis, S. H., Whitbeck, J. C., Krummenacher, C., Cohen, G. H., Eisenberg, R. J. & Wiley, D. C. (2001) Mol. Cell 8, 169-179.).

Entry activities of the complemented HSV-1 and HSV-2 viruses permit the conclusion that nectins are the principal entry receptors for most of the human cell lines tested, namely those of neuronal and epithelial origin. These cells could be infected with viruses complemented by wild-type gD or a mutant form of gD (Δ7-32) capable of using only nectin-1 as an entry receptor although, for HSV-2, entry activities were higher when the Q27P mutant (capable of using both nectin-1 and nectin-2) was used. In contrast, these cell lines fail to be infected by viruses complemented by gD mutants that are unable to infect cells via nectin-1 or nectin-2, but could use HVEM or (in the case of HSV-1) 3-O—S HS as entry receptors. Results obtained with the Jurkat cell line indicate that these cells of T lymphocyte origin can be infected via HVEM, consistent with their reported expression of HVEM (Zhai, Y., Guo, R., Hsu, T.-L., Yu, G.-L., Ni, J., Kwon, B. S., Jiang, G.-W., Lu, J., Tan, J., Ugustus, M., Carter, K., Rojas, L., Zhu, F., Lincoln, C., Endress, G., Xing, L., Wang, S., Oh, K.-O., Gentz, R., Ruben, S., Lippman, M. E., Hsieh, S.-L. & Yang, D. (1998) J. Clin. Invest. 102, 1142-1151.), or via one or both of the nectins. A potential caveat to these conclusions is the possibility that one or more HSV entry receptors remain undiscovered and that the mutations described here affect usage of these receptors similarly to the nectins, although an understanding of the mechanism is not necessary to practice the present invention and the present invention is not limited to any particular mechanism of action.

Experimental

The following examples are provided in order to demonstrate and further illustrate certain preferred embodiments and aspects of the present invention and are not to be construed as limiting the scope thereof.

Materials and Methods

Cell lines. Cell lines used included Chinese hamster ovary (CHO-K1) cells; CHO-K1 cells stably expressing human HVEM (Montgomery, R. I., Warner, M. S., Lum, B. J. & Spear, P. G. (1996) Cell 87, 427-436.), nectin-1 (Geraghty, R. J., Krummenacher, C., Cohen, G. H., Eisenberg, R. J. & Spear, P. G. (1998) Science 280, 1618-1620.) or nectin-2 (Warner, M. S., Geraghty, R. J., Martinez, W. M., Montgomery, R. I., Whitbeck, J. C., Xu, R., Eisenberg, R. J., Cohen, G. H. & Spear, P. G. (1998) Virology 246, 179-189.); a mutant baby hamster kidney cell line (BHK-95-19) (Roller, R. J. & Herold, B. C. (1997) J. Virol. 71, 5805-5813.); Vero cells; a Vero cell line carrying the HSV-1 gD gene (VD60) and used for the propagation of gD-negative HSV mutants (Ligas, M. W. & Johnson, D. C. (1988) J. Virol. 62, 1486-1494.); cell lines derived from human neuroblastomas, SH-SY5Y (Ross, R. A., Spengler, B. A. & Biedler, J. L. (1983) J Nat. Cancer Inst. 71, 741-747.) and IMR-5 (Gilbert, F. & Malenbaum, G. B. (1980) Adv. in Neuroblastoma Res., 59-72.); cell lines derived from carcinomas (C33A and A431); and Jurkat cells. The CHO cell line and derivatives were grown in Ham's F12 medium supplemented with 10% fetal bovine serum (FBS) and the Jurkat cells in RPMI-10% FBS. The other cell lines were grown in DMEM-10% FBS.

Virus strains. HSV-1(KOS)tk12 expresses β-galactosidase (β-gal) from an insert in the viral thymidine kinase gene (Warner, M. S., Geraghty, R. J., Martinez, W. M., Montgomery, R. I., Whitbeck, J. C., Xu, R., Eisenberg, R. J., Cohen, G. H. & Spear, P. G. (1998) Virology 246, 179-189). HSV-1(KOS)gD6 expresses β-gal from an insertion that replaces the gD gene (Warner, M. S., Geraghty, R. J., Martinez, W. M., Montgomery, R. I., Whitbeck, J. C., Xu, R., Eisenberg, R. J., Cohen, G. H. & Spear, P. G. (1998) Virology 246, 179-189). HSV-2(333)UL3/4Gal expresses β-gal from an insert between genes UL3 and UL4 that preserved the polyadenylation sites for both genes. This virus was made gD-negative by isolating an additional recombinant virus in which the gD open reading frame (ORF) was replaced by the gene for the enhanced green fluorescent protein (EGFP). To do this, VD60 cells were transfected with plasmid pSM152-GFP (see next section), using LipofectAMINE reagent (Invitrogen, Carslbad, Calif.), and then infected with HSV-2(333)UL3/4Gal at 5 plaque-forming units (PFU) per cell. Virus was isolated from fluorescent plaques and plaque-purified on VD60 cells. Nucleotide sequencing of polymerase chain reaction (PCR) products revealed that the gD ORF was replaced with the EGFP ORF as planned. The mutant, HSV-2(333)gD⁻Gal, had a titer of 3×10⁸ PFU/ml on VD60 cells and <10³ on Vero cells.

Plasmids. Plasmid pSM152-GFP was generated from plasmid pMY152, which contains the gD gene and portions of flanking genes (gJ and gI) from HSV-2(333) DNA (nucleotides 140487-143001 based on the published sequence of the related HSV-2(HG52) genome—GenBank # NC_{—}001798), cloned between the EcoRI and HindIII sites of pUC19. To generate pSM152-GFP, nucleotides 140978-141830 of the pMY152 insert were replaced with the EGFP open reading frame (ORF) from pEGFP-N1 (Clontech, Palo Alto, Calif.) so that its expression would be driven by the gD promoter. To generate HSV-1 mutant forms of gD, pDM20 was first constructed by PCR amplification of the HSV-1 (Patton) gD ORF from pCJ3 (30) and cleavage of the PCR product with HindIII (a natural site upstream of the gD ORF) and XhoI (a site added just after the gD stop codon) for cloning between HindIII and XhoI sites in pGEM 7Zf+ (Promega, Madison, Wis.). HSV-1 (Patton) gD is identical in sequence to HSV-1(KOS) gD except for a single amino acid substitution in the cleaved signal sequence. Nucleotide sequence analysis revealed that pDM20 had an unintended mutation (change of codon 215 from GAC to GGC) resulting in a D215G amino acid substitution (the first amino acid following the signal peptidase cleavage site is designated position 1). This mutation, in combination with others, proved to be associated with phenotypes of interest. The QuickChange site-directed mutagenesis kit (Stratagene, La Jolla, Calif.) was used to repair the mutation in codon 215, generating pDM60. Both pDM20 and pDM60 were used as template for QuickChange mutagenesis to introduce other nucleotide substitutions that resulted in amino acid substitutions, and then the HindIII/XhoI fragment containing the altered gD ORF was sub-cloned into pcDNA3 to generate expression plasmids pDM73 (D215G), pDM61 (Q132L), pDM27 (R222N), pDM29 (F223I), pDM23 (Q132L/D215G), pDM24 (S140N/D215G), pDM28 (R222N/D215G), pDM30 (F2231/D215G), pDM68 (R222N/F223I), and pDM80 (R222N/F223I/D215G). The pcDNA3-based plasmid expressing wild-type HSV-1 gD was pCJ3 (30). To introduce point mutations into the HSV-2 gD ORF, pMY1, which contains the wild-type HSV-2(333) gD ORF in pUC19 (Yoon, M., Zago, A., Shukla, D. & Spear, P. G. (2003) J. Virol. 77, 9221-9231.) was used as template for QuickChange mutagenesis. The DraIII-BspEI fragment containing the mutation(s) of interest was then excised and substituted for the wild-type HSV-2 gD fragment in pAZD2, which is a pCAGGS-based plasmid expressing wild-type HSV-2(333) gD (Zago, A. & Spear, P. G. (2003) J. Virol. 77, 9695-9699.), generating expression plasmids pSM03 (D215G), pSM01 (Q132L/D215G), pSM02 (S140N/D215G), pSM04 (R222N/D215G), pSM05 (F223I/D215G), pSM06 (R222N/F223I) and pSM07 (R222N/F223I/D215G). Regions of each HSV-1 gD ORF encoding the ectodomain were fused to the C-terminal 231 codons of the rabbit IgG heavy chain ORF by subcloning into pDM19 as described (Yoon, M., Zago, A., Shukla, D. & Spear, P. G. (2003) J. Virol. 77, 9221-9231.). The plasmids obtained expressed soluble secreted gD:Fc hybrid proteins. To generate similar hybrids for HSV-2 gD, the EcoRI/BamHI fragment from each of the pCAGGS-based plasmids named above was substituted for the equivalent fragment of pMY12, which expresses wild-type HSV-2 gD:Fc (22). The HSV-1 and HSV-2 constructs, respectively, were pMY80 and pMY12 (wild-type gD), pDM74 and pSM13 (D215G), pDM32 and pSM11 (Q132L/D215G), pDM33 and pSM12 (S140N/D215G), pDM38 and pSM14 (R222N/D215G), pDM40 and pSM15 (F2231/D215G), pDM69 and pSM16 (R222N/F2231), pCJDM85 and pSM17 (R222N/F2231/D215G). Plasmids expressing other mutant forms of HSV-1 and HSV-2 gD were pMY77 and pMY8 (Q27P), and pMY98 and pMY33 (A7-32) (Yoon, M., Zago, A., Shukla, D. & Spear, P. G. (2003) J. Virol. 77, 9221-9231.). Nucleotide sequencing of all plasmids confirmed the presence of desired mutations and absence of unintended mutations. All restriction enzymes were purchased from New England Biolabs (Ipswich, Mass.).

Preparation of gD:Fc protein. CHO-K1 cells were plated in 100 mm dishes and transfected with one of the plasmids expressing gD:Fc protein, using LipofectAMINE Plus (Invitrogen, Carlsbad, Calif.) according to the manufacturer's recommendations. After 5 hrs, 5 ml of DMEM-10% FBS was added to each dish for overnight incubation. The next day, the medium was replaced with Opti-MEM and the cells were incubated for an additional 24 hrs. The culture supernatants containing the secreted gD:Fc proteins were collected, clarified by low-speed centrifugation and concentrated 5× using Biomax filters (30 kD cut-off, Millipore (Billerica, Mass.). Concentrations of the gD:Fc proteins were determined by ELISA using an anti-rabbit Fc detection system and rabbit IgG for the standard curve.

Cell fusion assay. The transfection and assay conditions and plasmids used were as previously described (Zago, A. & Spear, P. G. (2003) J. Virol. 77, 9695-9699, Pertel, P., Fridberg, A., Parish, M. L. & Spear, P. G. (2001) Virology 279, 313-324.), except that the gD-expressing plasmids were those expressing the wild-type or mutant forms of HSV-1 or HSV-2 gD. CHO-K1 cells or BHK-95-19 cells (effector cells) were transfected with the HSV-1 or HSV-2 set of plasmids expressing the four glycoproteins required for cell fusion (gB, gD or a gD mutant, gH and gL) and T7 RNA polymerase (0.8 μg of the gB-expressing plasmid and 0.4 μg of each of the other plasmids per 6-well plate with LipofectAMINE 2000 for CHO cells or LipofectAMINE Plus for BHK-95-19 cells). Target cells included CHO-HVEM, CHO-nectin-1, and CHO-nectin-2 cells that had been transfected with a plasmid carrying the firefly luciferase gene under control of the T7 promoter (pT7ELCLuc). Other target cells included CHO-K1 cells co-transfected with pT7ELCLuc and a plasmid expressing 3-OST-3A and BHK-95-19 cells co-transfected with pT7ELCLuc and pBEC10 expressing HVEM (2), pBG38 expressing nectin-1 (Geraghty, R. J., Krummenacher, C., Cohen, G. H., Eisenberg, R. J. & Spear, P. G. (1998) Science 280, 1618-1620.), or pMW20 expressing nectin-2 (Warner, M. S., Geraghty, R. J., Martinez, W. M., Montgomery, R. I., Whitbeck, J. C., Xu, R., Eisenberg, R. J., Cohen, G. H. & Spear, P. G. (1998) Virology 246, 179-189.). At 6 hrs after transfection, the effector and target cells were trypsinized and replated at a 1:1 ratio in 96-well plates (5×10⁴ cells total per well for CHO and 2.5×10⁴ cells for BHK-95-19). After an additional 18 hrs, the cells were lysed and expression of luciferase was quantified using the luciferase assay kit (Promega, Madison, Wis.).

Cell ELISA assay. The binding of antibodies to cells expressing gD and of gD:Fc hybrids to cells expressing HSV entry receptors was quantitated by a cell ELISA (CELISA). For antibody binding, CHO-K1 cells were transfected with one of the gD-expressing plasmids described above and plasmids expressing T7 RNA polymerase and the other viral glycoproteins required for cell fusion. At 6 hrs after transfection, the cells were replated (without target cells) in 96-well plates. For gD:Fc binding, CHO-HVEM cells, CHO-nectin-1 cells, or CHO-K1 cells transfected with a plasmid expressing 3-OST-3A (Shukla, D., Liu, J., Blaiklock, P., Shworak, N. W., Bai, X., Esko, J. D., Cohen, G. H., Eisenberg, R. J., Rosenberg, R. D. & Spear, P. G. (1999) Cell 99, 13-22.) were plated directly into 96-well plates. After 18 hrs (or 42 hrs for the 3-OST-3A-transfected cells), the cells were washed with phosphate-buffered saline (PBS) and then incubated for 30 min with a rabbit polyclonal anti-gD serum, R7, at a 1:10,000 dilution in PBS-3% bovine serum albumen, or with serial dilutions of the culture supernatants containing the gD:Fc hybrids. Then, the cells were washed, fixed with PBS containing 2% formaldehyde and 0.2% glutaraldehyde, and incubated sequentially with biotinylated anti-rabbit IgG (Sigma, St. Lousi, Mo.), Amdex streptavidin-conjugated horseradish peroxidase (HRP, Amersham, Piscataway, N.J.), and HRP substrate (BioFx Lab, Owings Mills, Md.). Alternatively, the fixed cells were incubated sequentially with an HRP-coupled anti-rabbit Fc antibody and HRP substrate. HRP product was quantified at 380 nm in a Victor Wallac spectrophotometer (Perkin-Elmer, Boston, Mass.).

Interference assay. CHO-K1 cells were co-transfected with a plasmid expressing wild-type or mutant gD and pBG38 expressing human nectin-1 or pcDNA-3 as a control (4:1 ratio using 1.5 μg of plasmid DNA total per well with LipofectAMINE reagent in Opti-MEM). After 1 day, the cells were replated into 96-well plates. After an additional 12-24 hrs, the cells were exposed to serial dilutions of HSV-1(KOS)tk12. Six hrs later, the cells were washed, permeabilized and incubated with the β-gal substrate O-nitrophenyl-β-D-galactopyranoside (Sigma, St. Louis, Mo.) as described (Montgomery, R. I., Warner, M. S., Lum, B. J. & Spear, P. G. (1996) Cell 87, 427-436.) The reaction product was quantified at 405 nm as a measure of viral entry.

Complementation of gD-negative viruses for viral entry. Vero cells were transfected with plasmids expressing wild-type or mutant forms of gD using LipofectAMINE reagent or LipofectAMINE 2000. After 24 hrs, the cells were infected with the homologous complemented gd-negative virus, either HSV-1(KOS)gD6 (20 PFU per cell) or HSV-2(333)gD⁻Gal (10 PFU per cell). After 2 hrs, the virus inocula were removed and residual unpenetrated virus was inactivated by exposure of the cells to 0.1 M citrate buffer (pH 3.0) for 1 min. The cells were washed and incubated in growth medium for 24 hrs. To prepare virus stocks, the cells were then harvested and sonicated and the cell lysates centrifuged at low speed to remove debris. Various cell types grown in 96-well plates were inoculated with these stocks (70 μl per well) and incubated for 24 hrs. Viral entry was then assessed by quantifying the expression of β-gal as described above.

### EXAMPLE 1

Amino Acid Substitutions in HSV-1 and HSV-2 gD

The amino acid positions targeted for substitutions in gD included Q132, S140, R222 and F223, all of which are conserved between HSV-1 and HSV-2. Substitutions Q132L and S140N were found in HSV-1 mutants selected for resistance to neutralization by certain monoclonal antibodies (mAbs) (Muggeridge, M. I., Wu, T. T., Johnson, D. C., Glorioso, J. C., Eisenberg, R. J. & Cohen, G. H. (1990) Virology 174, 375-387.) that can block the binding of HSV-1 gD to both HVEM and nectin-1 (Krummenacher, C., Nicola, A. V., Whitbeck, J. C., Lou, H., Hou, W., Lambris, J. D., Geraghty, R. J., Spear, P. G., Cohen, G. H. & Eisenberg, R. J. (1998) J. Virol. 72, 7064-7074.). Substitutions R222N and F2231 were reported to reduce interactions with nectin-1 (Q. Bai, W. A. Shah, J. B. Cohen, R. J Eisenberg, G. H. Cohen and J. C. Glorioso, 2001, Abstract 2.10, 26^{th} International Herpesvirus Workshop, cited with permission from J. C. Glorioso, University of Pittsburgh), and also targeted a region where 3 amino acid deletions in HSV-1 gD had been shown to reduce entry activity with both HVEM and nectin-1 (Whitbeck, J. C., Muggeridge, M. I., Rux, A. H., Hou, W., Krumrnmenacher, C., Lou, H., van Geelen, A., Eisenberg, R. J. & Cohen, G. H. (1999) J. Virol. 73, 9879-9890.). Phenotypes of interest were observed as described below. Combinations of these substitutions with each other or with an unintended substitution, D215G, resulted in the novel phenotypes described here. The HSV-1 gD mutations analyzed included Q132L, D215G, R222N, F2231, Q132L/D215G, S140N/D215G, R222N/D215G, F223I/D215G, R222N/F223I, and R222N/F223I/D215G. The HSV-2 gD mutations analyzed included D215G and the double and triple mutations just named. Immunoassays showed that all the HSV-1 and HSV-2 gD mutants were essentially indistinguishable from wild-type HSV-1 or HSV-2 gD in cell surface expression (values obtained by CELISA ranged from 80-95% of wild-type controls).

### EXAMPLE 2

Effects of the gD Mutations on Binding to Entry/Fusion Receptors

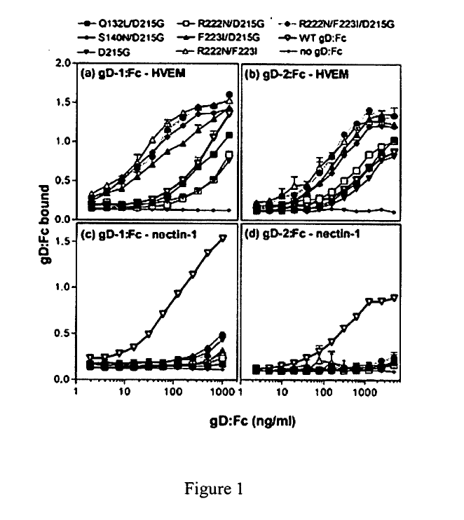

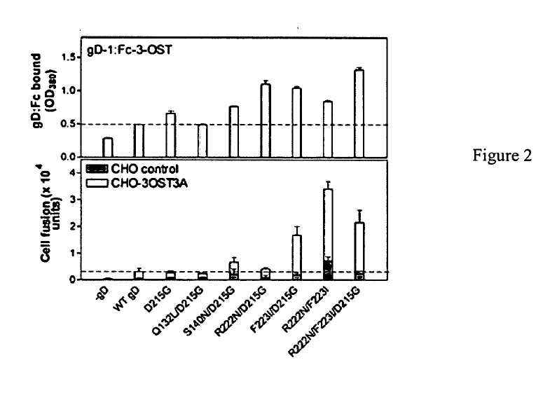

Two kinds of experiments were done to determine whether the mutant forms of gD could bind to the various entry receptors. First, CHO cells expressing HVEM, nectin-1 or 3-OST-3A were incubated with soluble forms of gD—hybrids of the gD ectodomains fused to the Fc region of rabbit IgG (gD:Fc hybrids)—and binding was quantified by CELISA. FIG. 1 shows the results of incubating serial dilutions of the wild-type and mutant forms of gD:Fc with CHO cells that stably express human HVEM or human nectin-1. Serial dilutions of concentrated culture supernatants containing known concentrations of the wild-type or mutant forms of HSV-1 gD:Fc (FIGS. 1A and 1C) or HSV-2 gD:Fc (FIGS. 1B and 1D) were incubated with confluent monolayers of CHO-HVEM cells (FIGS. 1A and 1B) or CHO-nectin-1 cells (FIGS. 1C and 1D) in 96-well format. After incubation apd washing, the cells were fixed and then cell-bound gD:Fc was quantified by an Fc detection system. The values presented (HRP reaction product detected at OD₃₈₀) are means and standard deviations of triplicate determinations and are representative of three independent experiments with similar results. All of the mutant gD:Fcs were severely impaired for binding to nectin-1 but retained ability to bind to HVEM (FIG. 1). Some of the mutant gD:Fcs (HSV-1 and HSV-2 forms of S140/D215G, F223I/D215G, R222N/F223I and R222N/F223I/D215G) bound to HVEM more efficiently than did the wild-type gD:Fcs. Two of the HSV-1 mutants (R222N/D215G and D215G) bound to HVEM somewhat less efficiently than wild-type. FIG. 2 (top panel) shows the binding of HSV-1 wild-type and mutant gD:Fcs to CHO-K1 cells expressing 3-OST-3A to generate 3-O-S HS. The top panel in FIG. 2 shows CHO-K1 cells that were transfected with a plasmid expressing 3-OST-3A, plated in 96-well dishes, and incubated with culture supernatants containing each of the forms of gD:Fc indicated (or no gD:Fc) at 1 μg/ml. Binding of the gD:Fcs to the cells was quantified and the results presented as described above with reference to FIG. 1. The bottom panel in FIG. 2 shows CHO-K1 cells that were co-transfected with plasmids expressing gB, gD (wild-type or mutant or empty vector for the −gD control), gH, gL and T7 polymerase, and were mixed 1:1 with CHO-K1 cells transfected with a plasmid expressing 3-OST-3A and a plasmid carrying the luceriferase gene under control of the T7 promoter or with CHO-K1 cells transfected with just the luciferase plasmid (control CHO cells). The cell mixtures were replated in 96-well dishes at 6 hrs after transfection. After 18 hrs of incubation, luciferase activity was quantified as a measure of cell fusion. The bars for control CHO-K1 cells are superimposed over the bars for the 3-OST-3A-expressing CHO-K1 cells to show the slightly enhanced fusion of the control cells observed with some mutant forms of HSV-1 gD. The values presented (luciferase activity in arbitrary units) are means and standard deviations of triplicate determinations and are representative of three independent experiments with similar results. Binding of wild-type gD:Fc was minimal, i.e., barely above the background due to the secondary detection reagents (−gD), but binding of some of the mutant gD:Fcs was significantly elevated, particularly for the R222N/D215G, F223I/D215G and R222N/F223I/D215G mutants. Binding studies were done with the HSV-2 gD:Fcs and nectin-2 but neither wild-type nor mutant forms of the gD:Fcs exhibited detectable binding as observed previously for wild-type (Lopez, M., Cocchi, F., Menotti, L., Avitabile, E., Dubreuil, P. & Campadelli-Fiume, G. (2000) J. Virol. 74, 1267-1274, Struyf, F., Martinez, W. M. & Spear, P. G. (2002) J. Virol. 76, 12940-12950.).

The second type of experiment was to test the ability of membrane-bound forms of wild-type and mutant gD to interact with nectin-1 in an interference assay. When cells are co-transfected with gD and a receptor to which it can bind, the cells can be as resistant to viral entry as if the receptor were not present at all, due to interfering interactions of the cell-associated gD with the receptor (Geraghty, R. J., Jogger, C. R. & Spear, P. G. (2000) Virology 268, 147-158.). CHO-K1 cells were co-transfected with a plasmid expressing human nectin-1 and plasmids expressing wild-type or mutant forms of gD or a control empty vector. After replating in 96-well plates, the cells were inoculated with an HSV-1 recombinant virus that expresses β-gal upon entry, HSV-1(KOS)tk12. Whereas wild-type gD caused severe interference with viral entry (entry was reduced to 10% of that observed for control cells co-transfected with the nectin-1 plasmid and empty vector), mutant R222N/F223I/D215G had no interfering activity. All the other mutants (D215G, Q132L/D215G, S140N/D215G, R222N/D215G, F223I/D215G, R222N/F223I) exhibited partial interfering activity, ranging from 40-60% of wild-type gD activity.

### EXAMPLE 3

Effects of the gD Mutations on Cell Fusion Activity with the Various Receptors

Two different cell types were used for cell fusion assays. BHK-95-19 cells were used to test functional activities of the HSV-1 and HSV-2 gD mutants because these cells lack entry/fusion receptors for both serotypes (Roller, R. J. & Herold, B. C. (1997) J. Virol. 71, 5805-5813.). CHO-K1 cells could also be used to test the HSV-1 gD mutants, but not the HSV-2 mutants, because CHO cells lack entry/fusion receptors for HSV-1 but have an endogenous receptor for HSV-2 (Zago, A. & Spear, P. G. (2003) J. Virol. 77, 9695-9699.) that gives a very high background level of cell fusion but not of viral entry. CHO-K1 cells had to be used to test functional activities of HSV-1 gD mutants with 3-O-S HS because they express the enzymes required for heparan sulfate modifications that must precede the action of 3-OST-3A (Shukla, D., Liu, J., Blaiklock, P., Shworak, N. W., Bai, X., Esko, J. D., Cohen, G. H., Eisenberg, R. J., Rosenberg, R. D. & Spear, P. G. (1999) Cell 99, 13-22.).

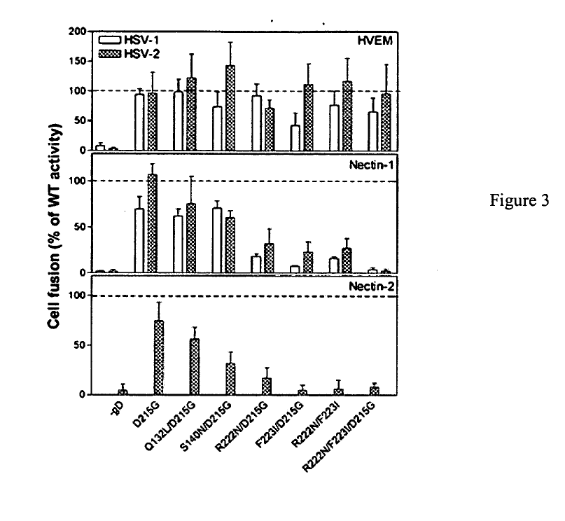

BHK-95-19 cells or CHO-K1 cells were co-transfected with plasmids expressing HSV-1 or HSV-2 gB, gD (mutant or wild-type), gH, gL and T7 polymerase (effector cells), and then detached and replated with a second population of BHK-95-19 (or CHO) cells that had been co-transfected with a plasmid expressing one of the HSV entry receptors and a plasmid carrying the luciferase gene under control of the T7 promoter (target cells). Controls included effector cells that received empty vector instead of a gD-expressing plasmid and target cells that received empty vector instead of a receptor-expressing plasmid. The cell fusion assay was performed as described above with reference to FIG. 2. The results for each mutant gD (or for the control with no gD) are normalized to the cell fusion activity observed for wild-type gD, set at 100%. Percent of wild-type cell fusion activity={(luciferase activity for mutant gD in the presence of receptor−luciferase activity for mutant gD in the absence of receptor)/(luciferase activity for wild-type gD in the presence of receptor−luciferase activity for wild type gD in the absence of receptor)}×100. The results shown are the means and standard deviations for at least three independent experiments, each done in triplicate. At 18 hrs after mixing and replating the effector and target cells, the cells were solubilized and substrate added for the quantification of luciferase activity, as a measure of cell fusion. The results presented in FIG. 3 (middle panel) show that cell fusion activity with nectin-1 as receptor was virtually abolished by the triple mutation, R222N/F223I/D215G, in either HSV-1 or HSV-2 gD. Also, cell fusion activity with nectin-1 was significantly reduced by the double mutations, R222N/D215G, F223I/D215G and R222N/F223I, especially for HSV-1 gD, whereas activity for the other three mutants (D215G, Q132L/D215G, S140N/D215G) was greater than 60% that of wild-type gD. These latter mutants supported the fusion of effector cells with nectin-1 target cells at levels higher than would have been predicted from the reduced binding activity shown in FIG. 1, but consistent with the interference results.

All the mutant forms of gD were active for fusion of effector cells with target cells expressing HVEM (FIG. 3, top panel), indicating that the mutant proteins were expressed and functional for cell fusion given an appropriate receptor. The levels of cell fusion activity observed ranged from about 75% to 125% of that associated with wild-type gD, except for HSV-1 mutant F2231/D215G, which was reduced to about 40% of wild-type activity. Results obtained with the HSV-2 mutants and target cells expressing nectin-2 were essentially the same as those obtained for target cells expressing nectin-1 (FIG. 3, middle and bottom panels). Although the wild-type form of HSV-1 gD was not very active in cell fusion with the receptor generated by 3-OST-3A in CHO-K1 cells (FIG. 2, lower panel), three of the mutations (double and triple mutations including the F2231 substitution) exhibited significantly enhanced fusion activity with target cells expressing 3-O-S HS, consistent with the gD:Fc binding data (FIG. 2, upper panel). Mutation R222N/F223I also enhanced cell fusion mediated by an endogenous receptor, perhaps the same CHO cell receptor that serves, inefficiently, as an entry/fusion receptor for HSV-2 (Zago, A. & Spear, P. G. (2003) J. Virol. 77, 9695-9699.).

Use of CHO-HVEM or CHO-nectin-1 cells to assess functional activities of the HSV-1 gD mutants in cell fusion gave results essentially the same as those shown for BHK-95-19 cells in FIG. 3, except that the mutants all exhibited wild-type or enhanced levels of fusion activity with HVEM. Three single amino acid substitutions in HSV-1 gD were also tested (Q132L, R222N, and F2231). None of these point mutations had significant effects on cell fusion activity with CHO-HVEM cells (activities observed were 90-130% of that observed for wild-type gD). Cell fusion activity with CHO-nectin-1 cells was greater than wild-type for Q312L and R222N (125 and 105%, respectively) and less (75%) for F2231.

### EXAMPLE 4

Effects of the gD Mutations on Viral Entry Via the Various Receptors

Wild-type gD or one of the mutant forms of gD was incorporated into the envelopes of gD-negative mutants of HSV-1 and HSV-2, so that the complemented viruses could be tested for ability to enter cells expressing various receptors. The complemented viruses were obtained by passing the gD-negative mutants once through Vero cells transfected to express either wild-type or mutant gD. Both viral mutants have a lacZ cassette inserted into the viral genome. Viral entry into cells bearing different entry receptors could therefore be compared by quantifying the amount of β-gal expressed at 24 hrs after inoculation. This measures the entry of input complemented virus because the mutant viruses generated from the first round of replication would lack gD and would be non-infectious.

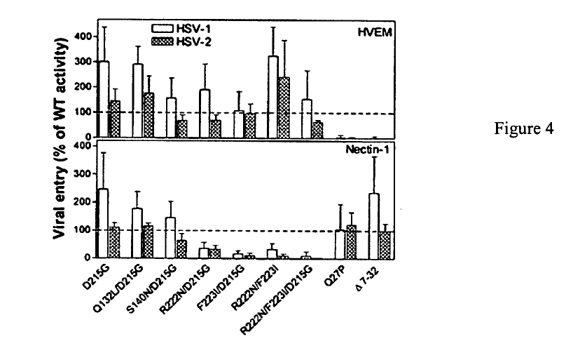

FIG. 4 shows that, for both HSV-1 and HSV-2, viruses complemented with the gD mutants having double or triple substitutions in positions D215, R222 and F223 were all significantly impaired for entry into CHO-nectin-1 cells, especially the triple mutant, whereas the other three mutants (D215G, Q132L/D215G, S140N/D215G) exhibited entry activities equivalent to, or higher than, that observed with wild-type gD. HSV-1 and HSV-2 gD-negative mutant viruses were passed through cells expressing each of the wild-type and mutant forms of gD so that the viral envelopes would incorporate the various forms of gD (complementation). Mutants Q27P and Δ7-32 were previously described (Yoon, M., Zago, A., Shukla, D. & Spear, P. G. (2003) J. Virol. 77, 9221-9231.). The complemented viruses, which can express β-gal from inserts in the viral genome, were used to inoculate CHO-HVEM and CHO-nectin-1 cells in 96-well dishes. After 24 hrs, the cells were lysed and β-gal activity (OD₄₅₀ for detection of the reaction product) quantified as a measure of viral entry. The results are normalized to the viral entry activity observed with wild-type gD, set at 100%. Percent of wild-type gD activity {(absorbance for mutant gD−absorbance in the absence of gD)/(absorbance for wild-type gD−absorbance in the absence of gD)}×100. The results shown are the means and standard deviations for at least three independent experiments, each done in triplicate. These results parallel the cell fusion results shown in FIG. 3. On the other hand, all the gD mutants just described were capable of mediating viral entry into cells expressing HVEM, at levels 75-300% of activity associated with wild-type gD. The other two gD mutants shown in FIG. 4 (Q27P and Δ7-32) were previously shown to retain fusion activity with nectin-1 but not with HVEM (Yoon, M., Zago, A., Shukla, D. & Spear, P. G. (2003) J. Virol. 77, 9221-9231.), a result confirmed here for viral entry as well. For each of the replicate experiments summarized in FIG. 4, a single set of fresh complemented virus preparations was used to inoculate both the CHO-HVEM cells and CHO-nectin-1 cells. Therefore, the failure of some mutants to infect cells via HVEM or nectin-1 was not due to failure of the mutant gDs to be incorporated into virions, inasmuch as the same complemented virus stock could infect cells via the other receptor.

### EXAMPLE 5

Effects of the gD Mutations on Viral Entry into Various Human Cell Lines

Human cell lines of neuronal, epithelial and lymphoid origin were selected for entry assays with complemented virus stocks. Two human cell lines derived from neuroblastomas (IMR-5 and SH-SY5Y), the cervical cancer cell line C33A, the vulvar carcinoma cell line A431 and the leukemic Jurkat T cell line were inoculated with the gd-deleted HSV-2 virus preparations complemented with the various HSV-2 gD mutants or wild-type gD. The neuroblastoma cell lines were also inoculated with the analogous complemented HSV-1 virus preparations. Expression of nectin-1 transcripts has been detected previously in both IMR-5 cells and SH-SY5Y cells and nectin-2 transcripts in SH-SY5Y cells (Geraghty, R. J., Krummenacher, C., Cohen, G. H., Eisenberg, R. J. & Spear, P. G. (1998) Science 280, 1618-1620, Warner, M. S., Geraghty, R. J., Martinez, W. M., Montgomery, R. I., Whitbeck, J. C., Xu, R., Eisenberg, R. J., Cohen, G. H. & Spear, P. G. (1998) Virology 246, 179-189.). Expression of cell surface nectin-1 has been detected on SH-SY5Y cells and A431 cells and of cell surface HVEM on IMR-5 cells, A431 cells and Jurkat cells (Krummenacher, C., Baribaud, F., Ponce de Leon, M., Baribaud, I., Whitbeck, J. C., Xu, R., Cohen, G. H. & Eisenberg, R. J. (2004) Virology 322, 286-299, Zhai, Y., Guo, R., Hsu, T.-L., Yu, G.-L., Ni, J., Kwon, B. S., Jiang, G.-W., Lu, J., Tan, J., Ugustus, M., Carter, K., Rojas, L., Zhu, F., Lincoln, C., Endress, G., Xing, L., Wang, S., Oh, K.-O., Gentz, R., Ruben, S., Lippman, M. E., Hsieh, S.-L. & Yang, D. (1998) J. Clin. Invest. 102, 1142-1151.).

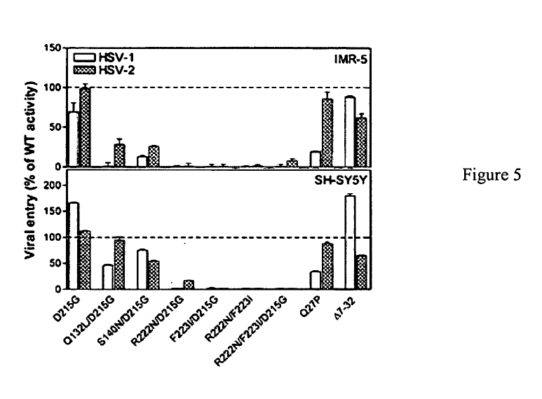

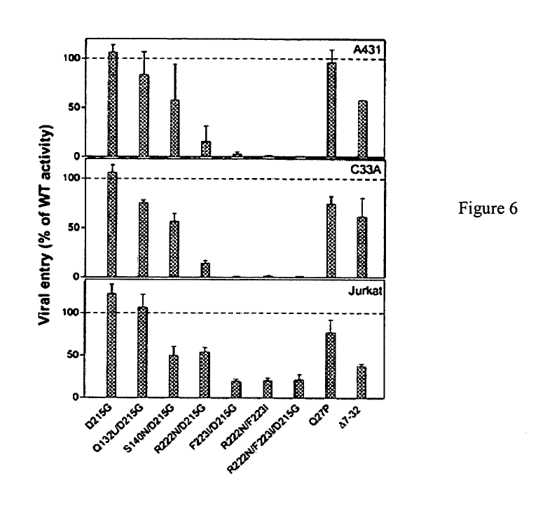

The results presented in FIGS. 5 and 6 demonstrate that viruses complemented with mutants F223I/D215G, R222N/F223I, and R222N/F223I/D215G were severely impaired in ability to infect both the neuroblastoma and epithelial cell lines, despite their ability to infect control CHO-HVEM cells at near wild-type efficiency (not shown), and despite reported expression of HVEM in some of these cells. These mutants were able to infect the Jurkat cells at about 25% of wild-type gD efficiency. FIG. 5 shows viral entry activities of the HSV-1 and HSV-2 gD mutants in human neuroblastoma cell lines, IMR-5 and SH-SY5Y. IMR-5 cells and SH-SY5Y were plated in 96-well dishes and inoculated with complemented viruses prepared as described above with reference to FIG. 4. Viral entry was quantified and the results normalized to viral entry activities obtained with wild-type gD, as described for FIG. 4, for two independent experiments. FIG. 6 shows viral entry activities of the HSV-2 gD mutants in two human epithelial cell lines, A431 and C33A, and in the Jurkat T cell line. The experiments were done as described above with reference to FIGS. 4 and 5. Viral entry was quantified and the results normalized to viral entry activities obtained with wild-type gD, as described for FIG. 4, for two independent experiments. The HSV-2 mutant R222N/D215G exhibited some viral entry activity (about 15% of wild-type) with the SH-SY5Y, A431, and C33A cells, and about 50% activity with the Jurkat cells. The other mutants (D215G, Q132L/D215G, S140N/D215G) exhibited viral entry activities ranging from about 50-100% of wild-type gD levels, except on IMR-5 cells. The Q132L/D215G and S1140N/D215G mutants were impaired for entry into IMR-5 cells, but not for entry into cells expressing high levels of nectin-1 (FIGS. 4 and 5). It has been noted previously that IMR-5 cells express considerably less nectin-1 than do SH-SY5Y cells, based on mRNA levels (Geraghty, R. J., Krummenacher, C., Cohen, G. H., Eisenberg, R. J. & Spear, P. G. (1998) Science 280, 1618-1620.), and quantitation of receptor levels on cell surfaces (Krummenacher, C., Baribaud, F., Ponce de Leon, M., Baribaud, I., Whitbeck, J. C., Xu, R., Cohen, G. H. & Eisenberg, R. J. (2004) Virology 322, 286-299.). The reduced levels of nectin-1 expression on IMR-5 cells may reveal impairment of the Q132L/D215G and S140N/D215G mutants for interactions with nectin-1, an impairment that was evident in the gD:Fc binding assays (FIG. 1).

HSV-1 and HSV-2 gD-negative viruses complemented with the gD mutants Q27P and Δ7-32 were able to infect all the human cell lines tested. The HSV-1, but not the HSV-2, form of Q27P had reduced ability to mediate viral entry into the neuroblastoma cells. The HSV-2 mutant Δ7-32 had reduced ability to mediate viral entry into the A431 and Jurkat cells. The Q27P mutants retain functional interactions with nectin-1 and nectin-2 whereas the Δ7-32 mutants are functional only with nectin-1 (Yoon, M., Zago, A., Shukla, D. & Spear, P. G. (2003) J. Virol. 77, 9221-9231.). Results obtained with the Jurkat cells indicate that HVEM (used by the double and triple point mutants), nectin-1 (used by Q27P and A7-32) and nectin-2 (used by Q27P) could all have a role in HSV-2 entry into these cells.

### EXAMPLE 6

Effects of Mutations in Herpes Simplex Virus Type 1 (HSV-1) gD on Viral Entry into Cells Expressing Three Different Viral Entry Receptors, HVEM, Nectin-1 or Nectin-2

Chinese hamster ovary cells expressing the receptors HVEM, nectin-1 or nectin-2 were plated in 96-well dishes and then inoculated with the indicated concentrations of HSV-1 (KOS)tk12/FRT gD expressing wild-type gD or various mutant forms of gD. These viruses were derived from HSV-1 (KOS)tk12/FRT, which expresses β-galactosidase from the lacZ gene inserted into the thymidine kinase (tk) locus and which has a Flp recombinase target (FRT) sequence inserted in place of the gD gene. The various wild-type and mutant alleles of gD were inserted by Flp recombinase at the position of the FRT site as described (Yoon M. and Spear PG. Proc. Nat.l Acad. Sci U.S.A. 101(49):17252-7, 2004 Dec. 7.). At 6 hrs after addition of virus, the cells were lysed and β-galactosidase quantified as a measure of viral entry, as described (Yoon M. and Spear P G. Proc. Nat.l Acad. Sci U.S.A. 101(49):17252-7, 2004 Dec. 7.). HSV-1 strains expressing wild-type gD can enter cells via HVEM or nectin-1, but not nectin-2. Mutation Q27P in HSV-1 gD abrogates entry via HVEM, but not nectin-1, and enhances entry via nectin-2. Mutation D30P abrogates entry via HVEM, but not nectin-1. The strains labeled 306.1, 307-1 and 307-2 are different clones of HSV-1 carrying mutation Y38R in gD. This mutation abrogates entry via nectin-1 but not HVEM.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A composition, comprising a herpes simplex virus wherein said herpes simplex virus expresses at least one amino acid substitution selected from the group consisting of substitutions at amino acid positions 38, 215, 222, and 223 of the glycoprotein D (gD) ectodomain.

2. The herpes simplex virus of claim 1, wherein said herpes simplex virus expresses two or more amino acid substitutions selected from the group consisting of substitutions at amino acid positions 215, 222, and 223 of the glycoprotein D (gD) ectodomain.

3. A method for eliciting an immune response in an organism, comprising:
a) providing an organism;
b) providing a herpes simplex virus wherein said herpes simplex virus expresses at least one amino acid substitution selected from the group consisting of substitutions at amino acid positions 38, 215, 222, and 223 of the glycoprotein D (gD) ectodomain; and
c) contacting said organism with said herpes simplex virus such that said organism manifests an immune response.

4. The herpes simplex virus of claim 3, wherein said herpes simplex virus expresses two or more amino acid substitutions selected from the group consisting of substitutions at amino acid positions 215, 222, and 223 of the glycoprotein D (gD) ectodomain.

5. A method for expressing a heterologous nucleic acid sequence in a cell, comprising:
d) providing a cell;
e) providing a herpes simplex virus vector wherein said herpes simplex virus vector expresses at least one amino acid substitution selected from the group consisting of substitutions at amino acid positions 27, 30, 38, 215, 222, and 223 of the glycoprotein D (gD) ectodomain;
f) providing a heterologous nucleic acid sequence; and
g) transfecting said cell with said herpes simplex virus vector containing said nucleic acid sequence such that said nucleic acid sequence is expressed in said cell.

6. The method of claim 5, wherein said cell is selected from the group consisting of an epithelial cell and a neuronal cell.

7. The method of claim 6, wherein said herpes simplex virus expresses at least one more amino acid substitutions selected from the group consisting of substitutions at amino acid positions 27 and 30 of the glycoprotein D (gD) ectodomain.

8. The method of claim 8, wherein said cell is selected from the group consisting of a lymphocyte and a leukocyte.

9. The method of claim 8, wherein said substitution of said amino acid is selected from the group consisting of amino acid substitutions at positions 38, 215, 222 and 223.

10. The method of claim 5, wherein said herpes simplex virus expresses two or more amino acid substitutions selected from the group consisting of substitutions at amino acid positions 215, 222, and 223 of the glycoprotein D (gD) ectodomain.
